(19) United States
(12) Patent Application Publication
(10) Pub. No.: US 2023/0094507 A1
(43) Pub. Date: Mar. 30, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa Kaneko, Kanagawa (JP); Tomohide Hiragami, Kanagawa (JP); Kenji Nagamiya, Kanagawa (JP); Yasuyuki Hosono, Kanagawa (JP); Nobuya Kitamura, Kanagawa (JP)

(21) Appl. No.: 17/929,758

(22) Filed: Sep. 6, 2022

(30) Foreign Application Priority Data
Sep. 27, 2021 (JP) 2021-157272

(51) Int. Cl. G06F 16/9538; G06F 16/9535

(52) U.S. Cl. CPC G06F 16/9538; G06F 16/9535

(57) ABSTRACT

An information processing apparatus includes at least one processor. The processor performs a process of acquiring input information, which is information input to the information processing apparatus, and biological information of a user, performing a web search based on at least one of the input information or the biological information in a case in which it is determined that an abnormality occurs in a physical condition of the user based on at least one of the input information or the biological information, and displaying a result of the web search.

### CROSS-REFERENCE TO RELATED APPLICATIONS

The present application claims priority under 35 U.S.C. § 119 to Japanese Patent Application No. 2021-157272, filed on Sep. 27, 2021. The above application is hereby expressly incorporated by reference, in its entirety, into the present application.

### BACKGROUND

### 1. Technical Field

The disclosed technology relates to an information processing apparatus, an information processing method, and an information processing program.

### 2. Description of the Related Art

The following technologies are known as technologies of providing useful information for health management of a user. For example, JP2016-184403A discloses that a computer extracts data based on user communication, specifies a problem related to the health from extracted data, generates a content indicating a first suggestion corresponding to the problem related to the health based on the extracted data, monitors the user communication, and generates an additional suggestion that is improvement on the first suggestion.

JP2016-170733A discloses that a computer stores information on a specific drug taken is stored in a storage unit in association with user identification information, and executes a process of posting a predetermined message related to the specific drug in a case in which it is detected that identification information indicating a posting source of a posted message corresponds to the user identification information stored in the storage unit, and it is detected that a predetermined keyword is included in the posted message.

### SUMMARY

In a case in which any abnormality occurs in his/her physical condition, it is common practice to search for a cause, a disease name, and a remedy by web search by himself/herself. However, it is difficult to acquire all the information requested by the user by one search, and it is necessary to repeat the web search while appropriately changing a search keyword. For example, in a case in which a user inputs a symptom, such as “I have a headache”, as a search keyword to be first input to a search engine, it is assumed that a website listing a plurality of causes that cause the symptom is extracted as a search result. In a case in which the user grasps the cause corresponding to the user, and then tries to acquire information on an optimal medicine in accordance with the cause or a nearby medical institution that provides a medical care in accordance with the cause by web search, a new search keyword needs to be input. It is troublesome to repeat the web search while appropriately changing the search keyword, and it is difficult for the user who is not accustomed to the web search to access necessary information.

The disclosed technology is made in view of the above points, and is to reduce a burden on a web search by the user in a case in which useful information for the health management of the user extracted by the web search is provided.

The disclosed technology relates to an information processing apparatus comprising at least one processor. The processor performs a process of acquiring input information, which is information input to the information processing apparatus, and biological information of a user, performing a web search based on at least one of the input information or the biological information in a case in which it is determined that an abnormality occurs in a physical condition of the user based on at least one of the input information or the biological information, and displaying a result of the web search.

The input information may include a search keyword input to a search engine. The input information may include a word transmitted or sent to a specified or unspecified party. The input information may include a word uttered by the user in a conversation with a chatbot operated on the information processing apparatus.

The processor may perform a process of performing a first web search using a word input as the input information as a first search keyword, and a second web search using a relation word related to the word input as the input information as a second search keyword, and displaying the result of the web search for each of the first web search and the second web search. The processor may derive the second search keyword by using an ontology database in which the first search keyword and the second search keyword are associated with each other.

The processor may perform a process of performing a third web search using a text generated based on the biological information as a third search keyword, and displaying a result of the third web search.

The processor may perform a process of classifying the results of the web search into a plurality of categories to display the classified results.

The disclosed technology relates to an information processing method executed by at least one processor provided in an information processing apparatus, the method comprising acquiring input information, which is information input to the information processing apparatus, and biological information of a user, performing a web search based on at least one of the input information or the biological information in a case in which it is determined that an abnormality occurs in a physical condition of the user based on at least one of the input information or the biological information, and displaying a result of the web search.

The disclosed technology relates to an information processing program causing at least one processor provided in an information processing apparatus to execute a process comprising acquiring input information, which is information input to the information processing apparatus, and biological information of a user, performing a web search based on at least one of the input information or the biological information in a case in which it is determined that an abnormality occurs in a physical condition of the user based on at least one of the input information or the biological information, and displaying a result of the web search.

According to the disclosed technology, it is possible to reduce the burden on the web search by the user in a case in which useful information for the health management of the user extracted by the web search is provided.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a health management system according to an embodiment of the disclosed technology;

FIG. 2 is a diagram showing an example of a hardware configuration of an information processing apparatus according to the embodiment of the disclosed technology;

FIG. 3 is a functional block diagram showing an example of a functional configuration of the information processing apparatus according to the embodiment of the disclosed technology;

FIG. 4 is a functional block diagram showing an example of a functional configuration in a case in which a search processing unit according to the embodiment of the disclosed technology performs a web search based on input information;

FIG. 5A is a diagram showing an example of data recorded in a relation word database according to the embodiment of the disclosed technology;

FIG. 5B is a diagram showing another example of a recording format of the data recorded in the relation word database according to the embodiment of the disclosed technology;

FIG. 6 is a functional block diagram showing an example of a functional configuration in a case in which the search processing unit according to the embodiment of the disclosed technology performs the web search based on biological information;

FIG. 7 is a diagram showing an example of a display form of a result of the web search in a case in which the web search is performed based on the input information according to the embodiment of the disclosed technology;

FIG. 8 is a diagram showing another example of the display form of the result of the web search in a case in which the web search is performed based on the input information according to the embodiment of the disclosed technology;

FIG. 9 is a flowchart showing an example of a flow of a process performed by executing a search program according to the embodiment of the disclosed technology;

FIG. 10 is a flowchart showing details of a second web search process according to an embodiment of the disclosed technology;

FIG. 11 is a flowchart showing details of a third web search process according to the embodiment of the disclosed technology; and

FIG. 12 is a diagram showing an example of a display form of a result of a web search according to the embodiment of the disclosed technology.

### DETAILED DESCRIPTION

In the following, an example of an embodiment of the disclosed technology will be described with reference to the drawings. It should be noted that the same or equivalent components and portions in the drawings are represented by the same reference numerals, and the overlapping description will be omitted.

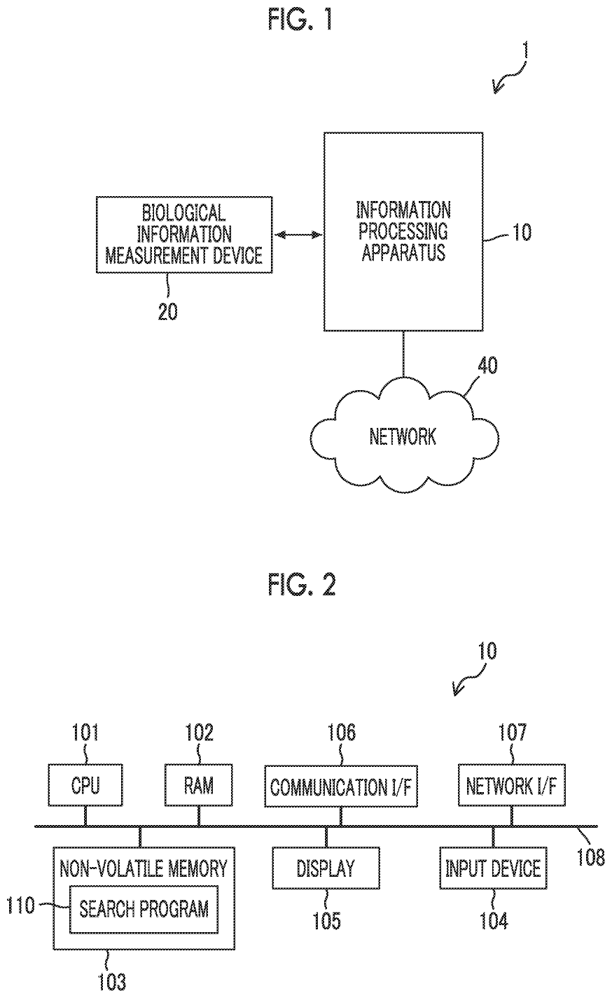

FIG. 1 is a diagram showing an example of a configuration of a health management system 1 according to an embodiment of the disclosed technology. The health management system 1 includes an information processing apparatus 10 and a biological information measurement device 20.

The information processing apparatus 10 is a communication terminal apparatus used by a user who is a management target subject by the health management system 1. The information processing apparatus 10 may be, for example, a desktop computer, a laptop, a tablet computer, or a smartphone. The information processing apparatus 10 is connected to a network 40 that can be used by the public, such as the Internet. The information processing apparatus 10 can access a web server (not shown) via the network 40, or can perform a web search by using a search engine.

The biological information measurement device 20 measures biological information of the user. The biological information may be, for example, information indicating at least one of a body temperature, a heartbeat, electrocardiography, myoelectricity, a blood pressure, arterial oxygen saturation (SpO2), a blood glucose level, or a lipid level. The biological information measurement device 20 may be a wearable device, such as a smart watch, comprising a sensor that measures these pieces of the biological information. The biological information measurement device 20 has a function of continuously measuring the biological information of the user in accordance with a predetermined time schedule and storing the biological information in an internal memory. The biological information measurement device 20 may measure the biological information for each predetermined period (for example, at intervals of 30 minutes). The biological information measurement device 20 is communicably connected to the information processing apparatus 10, and transmits the measured biological information to the information processing apparatus 10.

FIG. 2 is a diagram showing an example of a hardware configuration of the information processing apparatus 10. The information processing apparatus 10 includes a central processing unit (CPU) 101, a random access memory (RAM) 102, a non-volatile memory 103, an input device 104 including a keyboard, a mouse, a microphone, and the like, a display 105, a communication interface 106, and a network interface 107. The hardware is connected to a bus 108.

The display 105 may be a touch panel display. The communication interface 106 is an interface for the information processing apparatus 10 to perform communication with the biological information measurement device 20. A communication method may be any of wired or wireless. For wireless communication, for example, a method compliant with an existing wireless communication standard, such as Wi-Fi (registered trademark) and Bluetooth (registered trademark), can be applied. The network interface 107 is an interface for the information processing apparatus 10 to be connected to the network 40. A communication method may be any of wired or wireless. For wireless communication, for example, a method compliant with an existing wireless communication standard, such as Wi-Fi (registered trademark), can be applied.

The non-volatile memory 103 is a storage medium, such as a hard disk and a flash memory, and stores a search program 110. The RAM 102 is a work memory for the CPU 101 to execute a process. The CPU 101 loads the search program 110 stored in the non-volatile memory 103 into the RAM 102, and executes the process in accordance with the search program 110. The CPU 101 is an example of a “processor” according to the disclosed technology.

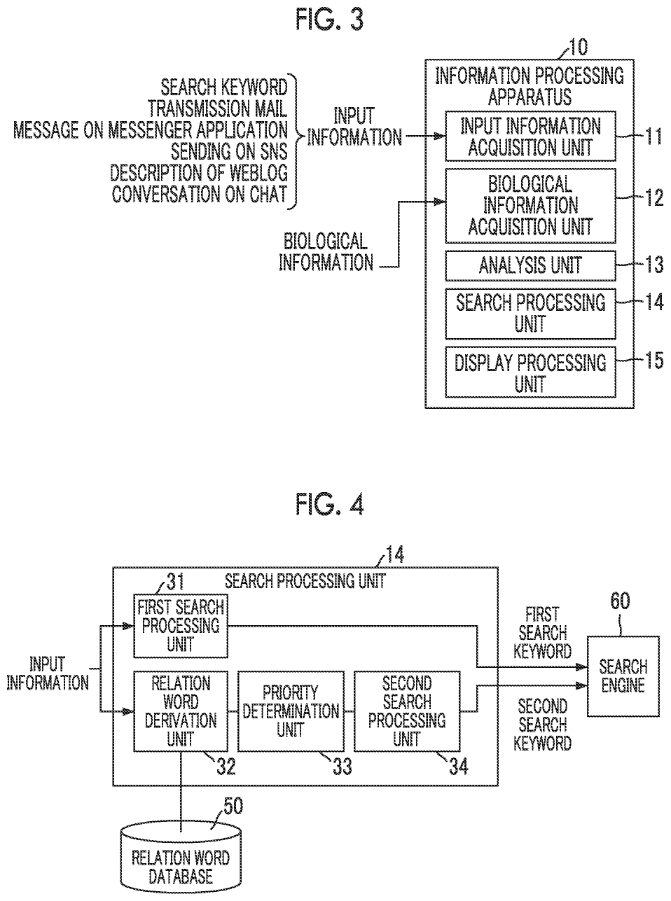

FIG. 3 is a functional block diagram showing an example of a functional configuration of the information processing apparatus 10. The information processing apparatus 10 includes an input information acquisition unit 11, a biological information acquisition unit 12, an analysis unit 13, a search processing unit 14, and a display processing unit 15. By the CPU 101 executing the search program 110, the CPU 101 functions as the input information acquisition unit 11, the biological information acquisition unit 12, the analysis unit 13, the search processing unit 14, and the display processing unit 15.

The input information acquisition unit 11 acquires input information input to the information processing apparatus 10. The input information may include a word input to the information processing apparatus 10. The input of the word as the input information can be performed by using, for example, the keyboard or the microphone as the input device 104. The input information may be information input on various applications installed in the information processing apparatus 10.

For example, the input information may include a search keyword input to a search engine for searching for information on a network. In addition, the input information may include a word transmitted or sent to a specified or unspecified party, such as a transmission mail, a message on a messenger application, sending on social networking service (SNS), description of a weblog, or a chat conversation. In addition, the input information may include a word uttered by the user in a conversation with a chatbot operated on the information processing apparatus 10. The input information acquisition unit 11 may acquire a combination of any two or more of information described above as the input information.

The input information acquisition unit 11 acquires the input information by linking with various applications, such as the web browser, an e-mail application, a messenger application, and an SNS application installed in the information processing apparatus 10.

The biological information acquisition unit 12 acquires the biological information of the user measured by the biological information measurement device 20. The biological information acquisition unit 12 acquires the measurement value of the body temperature, the heartbeat, the electrocardiography, the myoelectricity, the blood pressure, the arterial oxygen saturation (SpO2), the blood glucose level, the lipid level, or the like as the biological information from the biological information measurement device 20 by the communication with the biological information measurement device 20. In a case in which the biological information is intermittently measured by the biological information measurement device 20, the biological information acquisition unit 12 may acquire the biological information each time the measurement is performed by the biological information measurement device 20. In a case in which the biological information is time-series data, such as the electrocardiographic waveform, the biological information acquisition unit 12 may acquire the time-series data over a predetermined period (for example, 1 minute) as the biological information in one measurement. In addition, in a case in which the time-series data, such as the electrocardiographic waveform, is always measured by the biological information measurement device 20, the biological information acquisition unit 12 may always acquire the time-series data.

The analysis unit 13 analyzes the input information acquired by the input information acquisition unit 11 and the biological information acquired by the biological information acquisition unit 12, and determines whether or not an abnormality occurs in a physical condition of the user based on the input information and the biological information. The analysis unit 13 may determine that the abnormality occurs in the physical condition of the user in a case in which, for example, a word, which is registered in advance as a word related to the physical condition, a symptom, health, feeling, emotion, or a disease name of the user, is included in the word as the input information.

For example, in a case in which the input information is the search keyword input to the search engine, in a case in which the word registered in advance, such as “fever”, “cough”, “headache”, “stomach ache”, “dizzy”, “cold”, “I cannot sleep”, “anxiety”, “I feel depressed”, “I feel listless”, “I get annoyed”, “influenza”, “novel coronavirus”, or “COVID-19” is included in the search keyword, the analysis unit 13 may determine that the abnormality occurs in the physical condition of the user. In addition, for example, in a case in which the input information is the message input on the messenger application, in a case in which the registered word described above is included in the message, the analysis unit 13 may determine that the abnormality occurs in the physical condition of the user.

In addition, in a case in which the measurement value of the body temperature, the heartbeat, the electrocardiography, the myoelectricity, the blood pressure, the arterial oxygen saturation (SpO2), the blood glucose level, the lipid level, or the like as the biological information is not within a predetermined range, the analysis unit 13 may determine that the abnormality occurs in the physical condition of the user. The predetermined range described above may be a predetermined fixed value, or may be a range determined based on the measurement value of the user in a most recent predetermined period. In addition, in a case in which a fluctuation amount of the measurement value as the acquired biological information is larger than a predetermined amount, the analysis unit 13 may determine that the abnormality occurs in the physical condition of the user. The fluctuation amount described above may be, for example, a fluctuation amount from an average of the measurement values of the user in the most recent predetermined period (for example, one week).

In a case in which it is determined that the abnormality occurs in the physical condition of the user based on at least one of the input information or the biological information, the search processing unit 14 performs the web search based on at least one of the input information or the biological information. That is, in a case in which it is determined that the abnormality occurs in the physical condition of the user based only on the input information out of the input information and the biological information, the search processing unit 14 performs the web search based on the input information. In addition, in a case in which it is determined that the abnormality occurs in the physical condition of the user based only on the biological information out of the input information and the biological information, the search processing unit 14 performs the web search based on the biological information. In addition, in a case in which it is determined that the abnormality occurs in the physical condition of the user based on each of the input information and the biological information, the search processing unit 14 performs the web search based on both the input information and the biological information.

In a case in which the search processing unit 14 performs the web search based on the input information, the search processing unit 14 performs a first web search using a word input as the input information as a first search keyword. The search processing unit 14 further performs a second web search using a relation word related to the word input as the input information as a second search keyword.

FIG. 4 is a functional block diagram showing an example of a functional configuration in a case in which the search processing unit 14 performs the first and second web searches based on the input information. The search processing unit 14 includes a first search processing unit 31, a relation word derivation unit 32, a priority determination unit 33, and a second search processing unit 34.

The first search processing unit 31 performs the first web search by inputting the word input as the input information to a search engine 60 as the first search keyword. For example, the first search processing unit 31 inputs, as the input information, the word of “I have a headache” sent from the user on the messenger application to the search engine 60 as the first search keyword. It should be noted that the search engine 60 may be present on a website (so-called search site) accessible via the network 40.

The relation word derivation unit 32 derives one or more relation words related to the word input as the input information. The relation word derivation unit 32 derives the relation word by referring to a relation word database 50. The relation word database 50 is a database in which a word that can be input as the input information (hereinafter, referred to as an input word) and a relation word that can be used as the search keyword for the web search are recorded in association with the input word. The relation word database 50 may be stored in the non-volatile memory 103, or may be stored in an external storage (not shown) accessible via the network 40.

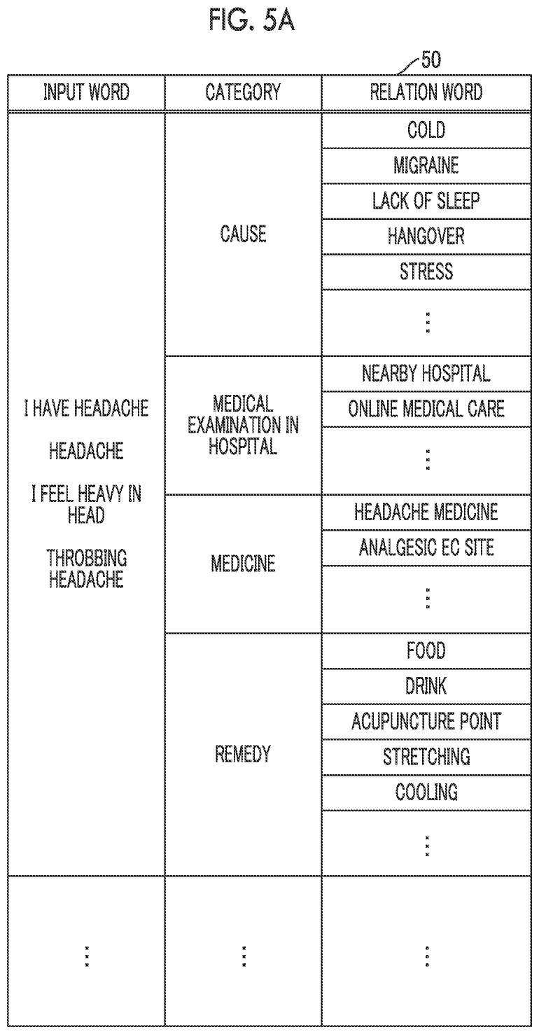

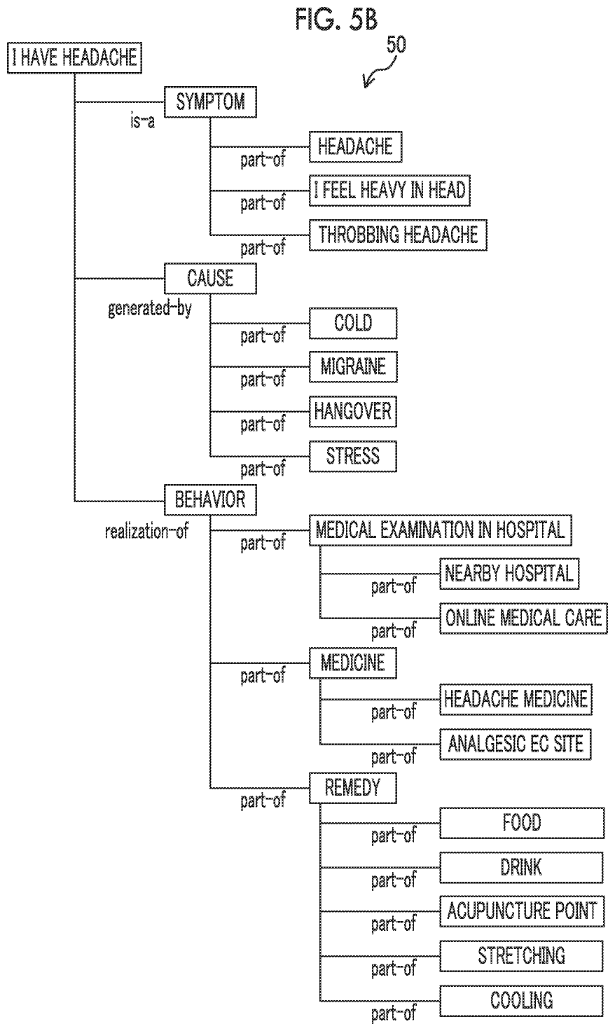

FIG. 5A is a diagram showing an example of data recorded in the relation word database 50. FIG. 5A shows a plurality of relation words associated with the input words of “I have a headache”, “headache”, “I feel heavy in the head”, and “throbbing headache”. The plurality of relation words are classified by a plurality of categories determined in accordance with the input word. In the example shown in FIG. 5A, the categories corresponding to the input words of “I have a headache”, “headache”, “I feel heavy in the head”, and “throbbing headache” are “cause”, “medical examination in a hospital”, “medicine”, and “remedy” are shown. In addition, FIG. 5A shows “cold”, “migraine”, “lack of sleep”, “hangover”, and “stress” are shown as the relation words classified into “cause”, and “nearby hospital” and “online medical care” as the relation words classified into “medical examination in a hospital”. In addition, “headache medicine” and “analgesic EC site” are shown as the relation words classified into “medicine”, and “food”, “drink”, “acupuncture point”, “stretching”, and “cooling” are shown as the relation words classified into “remedy”. It should be noted that the categories and the relation words corresponding to the input words may be updated at any time based on the search result. The relation word derivation unit 32 derives the relation word related to the input word for each of the plurality of categories by referring to the relation word database 50.

In addition, the relation word database 50 may be associated with the relation word in an ontological format as shown in FIG. 5B. The ontology is knowledge that defines the meaning of a concept used in a case of describing a model or knowledge about the target world, their usage, and conventions, and is a conceptual system that connects a plurality of concepts. FIG. 5B shows an ontology system diagram that systematizes related concepts from “I have a headache”. In the example shown in FIG. 5B, “headache”, “I feel heavy in the head”, and “throbbing headache” are associated with “I have a headache” as similar expressions. In a case in which these similar expressions are input as the input words, the similar expressions are treated as synonymous with “I have a headache”. In addition, “cold”, “migraine”, “lack of sleep”, “hangover”, and “stress” are associated as “cause” of “I have a headache”. In addition, “medical examination in a hospital”, “medicine”, and “remedy” are associated with “behavior” for “I have a headache”. In addition, a plurality of items are subordinate to the behavior. By defining such a phylogenetic relationship, the classification of the relation words and specific examples are organized. In addition, even in a case in which the input words are different, the expandability can be enhanced by associating the input words in the ontology format. It should be noted that “is-a”, “part-of”, “generated-by”, and “realization-of” described between the concepts in FIG. 5B represent a relationship between the concepts. “is-a” means an upper-lower relationship, “part-of” means a partial relationship, “generated-by” means an upper operation relationship, and “realization-of” means a lower operation relationship.

The priority determination unit 33 assigns priorities to the plurality of relation words derived by the relation word derivation unit 32. The priority determination unit 33 may assign the priorities to the plurality of relation words derived by the relation word derivation unit 32, for example, based on a frequency of use with the corresponding input words. The frequency of use may be a frequency of use by the user or may be a frequency of use by an unspecified number of people. The priority determination unit 33 assigns the priority to each of the relation words classified into the categories for each category of the relation words.

The second search processing unit 34 performs the second web search by extracting one or two or more relation words having relatively high priorities assigned by the priority determination unit 33 for each category, and inputting each relation word extracted for each category to the search engine as the second search keyword. The second search processing unit 34 may perform the second web search by inputting a combination of the first search keyword and the second search keyword to the search engine. In addition, the second search processing unit 34 may perform the second web search for each category. That is, the second web search can be performed a plurality of times.

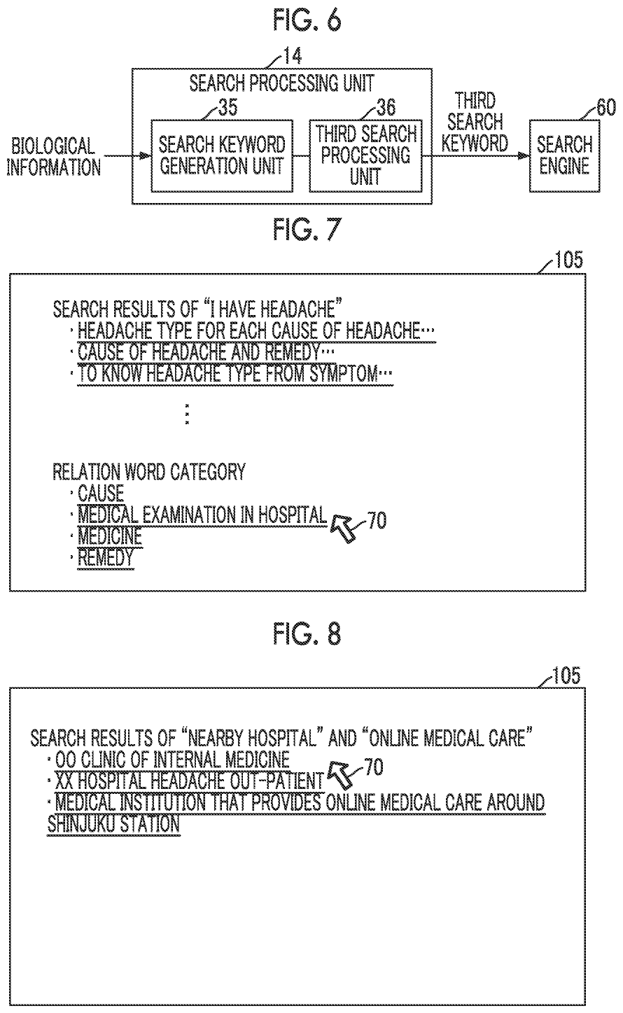

In a case in which the web search is performed based on the biological information, the search processing unit 14 performs a third web search using a text generated based on the biological information as a third search keyword. FIG. 6 is a functional block diagram showing an example of a functional configuration in a case in which the search processing unit 14 performs the web search based on the biological information. The search processing unit 14 includes a search keyword generation unit 35 and a third search processing unit 36.

The search keyword generation unit 35 generates the third search keyword for performing the web search based on the measurement value of the body temperature, the heartbeat, the electrocardiography, the myoelectricity, the blood pressure, the arterial oxygen saturation (SpO2), the blood glucose level, the lipid level, or the like, as the biological information. For example, the search keyword generation unit 35 may generate a text describing the type of the biological information and the measurement value, such as “body temperature 39 point 2 degrees” as the third search keyword, and may generate a text indicating a qualitative symptom specified from various measurement values, such as “high heart rate and rapid increase in blood glucose level” as the third search keyword. The third search processing unit 36 performs the third web search by inputting the third search keyword generated by the search keyword generation unit 35 to the search engine.

It should be noted that, in a case in which the web search is performed based on both the input information and the biological information, the search processing unit 14 may perform the web search using a combination of the first search keyword and the third search keyword. In addition, the search processing unit 14 may further perform the web search using a combination of the second search keyword and the third search keyword. In addition, the first to third web searches may be performed independently.

The display processing unit 15 performs a process of displaying, on the display 105, the result of the web search performed by the search processing unit 14. FIGS. 7 and 8 are diagrams showing examples of a display form of the result of the web search in a case in which the web search is performed based on the input information.

The display processing unit 15 performs a process of displaying a list of links for accessing the website extracted by performing the first web search on the display 105 as the result of the web search. FIG. 7 shows a state in which links to a plurality of websites extracted by the first web search using the word of “I have a headache”, which is the input word, as the first search keyword are displayed on the display 105.

The display processing unit 15 classifies the results of the second web search into the plurality of categories and displays the classified results. The display processing unit 15 displays, on the display 105, “cause”, “medical examination in a hospital”, “medicine”, and “remedy” as the categories for classifying the second search keywords (relation words) together with the result of the first web search. In a case in which any of the plurality of categories displayed on the display 105 is selected by using a mouse pointer 70, the display processing unit 15 displays, on the display 105, the links to the plurality of websites extracted by the second web search using the relation word classified into the selected category as the second search keyword. FIG. 8 shows a state in which “medical examination in a hospital” is selected from among the plurality of categories, and links to a plurality of websites extracted by the second web search using the words of “nearby hospital” and “online medical care” which are the relation words classified into “medical examination in a hospital” as the second search keyword are displayed on the display 105.

In a case in which the web search (third web search) is performed based on the biological information, the display processing unit 15 performs a process of displaying, on the display 105, the result of the third web search alone or together with the result of the web search (first and second web searches) based on the input information, depending on whether or not the web search (first and second web searches) based on the input information is performed.

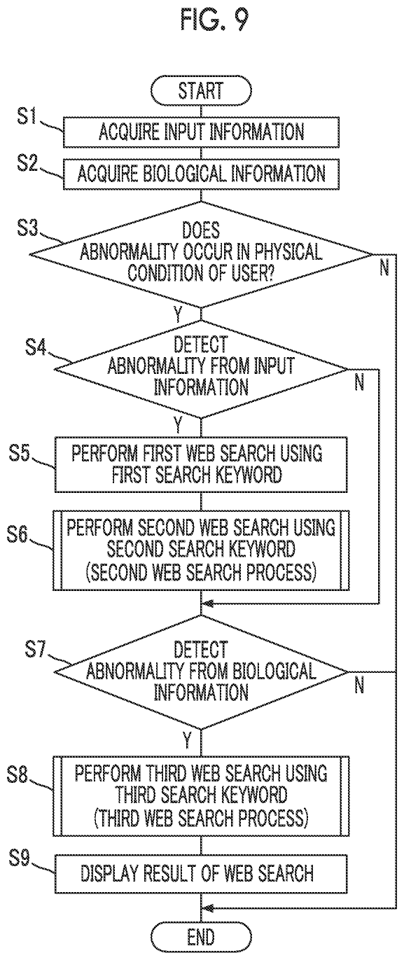

FIG. 9 is a flowchart showing an example of a flow of a process performed by the CPU 101 executing the search program 110. The search program 110 is executed, for example, in a case in which the user gives an instruction to start the process by operating the input device 104.

In step S1, the CPU 101 functions as the input information acquisition unit 11 and acquires the input information which is information input to the information processing apparatus 10.

In step S2, the CPU 101 functions as the biological information acquisition unit 12 and acquires the biological information from the biological information measurement device 20.

In step S3, the CPU 101 functions as the analysis unit 13 and determines whether or not the abnormality occurs in the physical condition of the user based on the input information acquired in step S1 and the biological information acquired in step S2. In a case in which it is determined that the abnormality occurs in the physical condition of the user, the process proceeds to step S4, and in a case in which it is determined that the abnormality does not occur in the physical condition of the user, the present routine ends.

In step S4, the CPU 101 determines whether or not the abnormality in the physical condition of the user is detected from the input information. In a case in which it is determined that the abnormality in the physical condition of the user is detected from the input information, the process proceeds to step S5, and in a case in which it is determined that the abnormality in the physical condition of the user is not detected from the input information, the process proceeds to step S7.

In step S5, the CPU 101 functions as the search processing unit 14 (first search processing unit 31) and performs the first web search using the word input as the input information as the first search keyword.

In step S6, the CPU 101 functions as the search processing unit 14 and performs the second web search using the relation word related to the word input as the input information as the second search keyword.

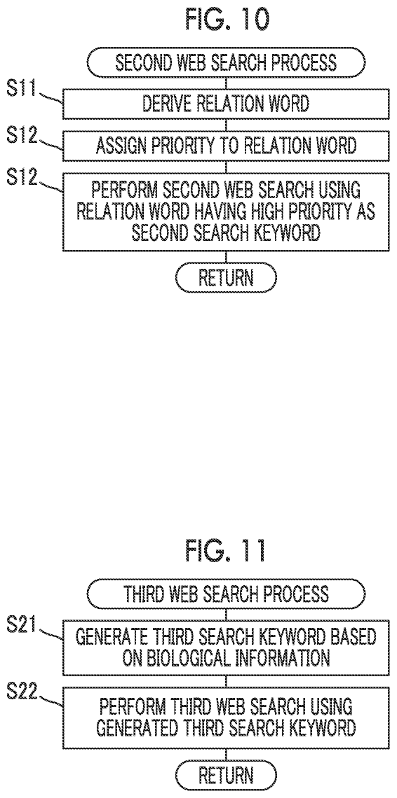

FIG. 10 is a flowchart showing the details of a second web search process in step S6. In step S11, the CPU 101 functions as the relation word derivation unit 32 and derives the relation word related to the word input as the input information. The CPU 101 derives one or more relation words for each of the plurality of categories by referring to the relation word database 50.

In step S12, the CPU 101 functions as the priority determination unit 33 and assigns the priorities to the plurality of relation words derived in step S11. The CPU 101 assigns the priority to each of the relation words classified into the categories for each category in the relation word database 50.

In step S13, the CPU 101 functions as the second search processing unit 34, extracts one or two or more relation words having relatively high priorities for each category, and inputs the relation words extracted for each category to the search engine as the second search keyword, thereby performing the second web search.

With reference to FIG. 9, in step S7, the CPU 101 determines whether or not the abnormality in the physical condition of the user is detected from the biological information. In a case in which it is determined that the abnormality in the physical condition of the user is detected from the biological information, the process proceeds to step S8, and in a case in which it is determined that the abnormality in the physical condition of the user is not detected from the biological information, the present routine ends.

In step S8, the CPU 101 functions as the search processing unit 14 and performs the third web search using the third search keyword generated based on the biological information.

FIG. 11 is a flowchart showing the details of a third web search process in step S8. In step S21, the CPU 101 functions as the search keyword generation unit 35 and generates the third search keyword for performing the web search based on the measurement value of the body temperature, the heartbeat, the electrocardiography, the myoelectricity, the blood pressure, the arterial oxygen saturation (SpO2), the blood glucose level, the lipid level, or the like, as the biological information.

In step S22, the CPU 101 functions as the third search processing unit 36 and inputs the third search keyword generated in step S21 to the search engine, thereby performing the third web search.

With reference to FIG. 9, in step S9, the CPU 101 functions as the display processing unit 15 and performs the process of displaying, on the display 105, the result of the web search by the search processing unit 14. In a case in which the web search based on the input information is performed, the CPU 101 classifies the results of the second web search using the relation words into the plurality of categories and displays the classified results.

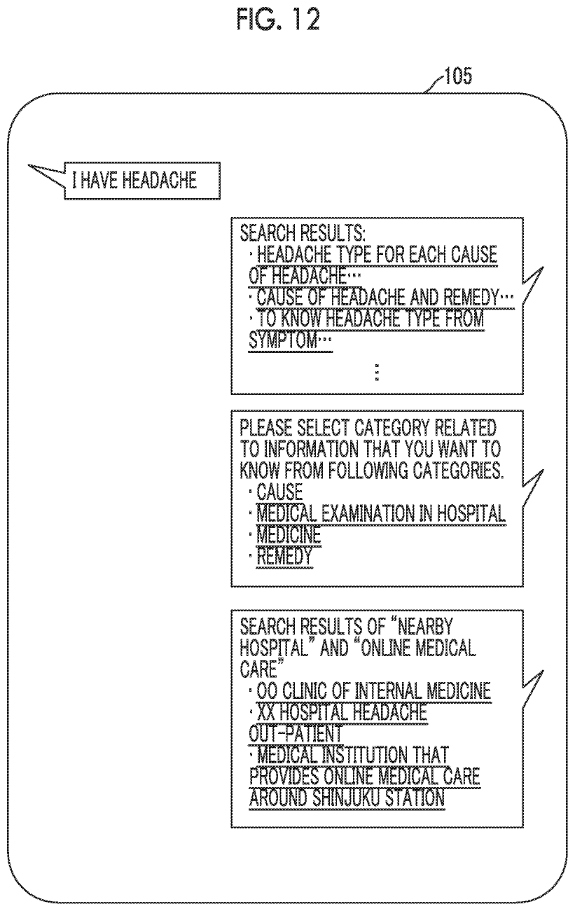

It should be noted that the input of the input information and the presentation of the result of the web search may be performed on a conversation screen with the chatbot which is operated on the information processing apparatus 10, as shown in FIG. 12.

FIG. 12 shows a case in which the user utters the word of “I have a headache” to the chatbot. In response to this, the chatbot displays, on the conversation screen, the links to the plurality of web sites extracted by inputting the word of “I have a headache” to the search engine as the first search keyword. In addition, the chatbot displays “cause”, “medical examination in a hospital”, “medicine”, and “remedy” as the categories for classifying the relation words, which are the second search keywords, on the conversation screen.

In a case in which, for example, “medical examination in a hospital” is selected by the user from among the categories displayed on the conversation screen, the chatbot displays, on the conversation screen, links to the plurality of websites extracted by inputting the words of “nearby hospital” and “online medical care” which are the relation words classified into “medical examination in a hospital” to the search engine as the second search keyword.

As described above, the information processing apparatus 10 according to the embodiment of the disclosed technology performs a process of acquiring input information, which is information input to the information processing apparatus, and biological information of a user, performing a web search based on at least one of the input information or the biological information in a case in which it is determined that an abnormality occurs in a physical condition of the user based on at least one of the input information or the biological information, and displaying a result of the web search. As described above, with the information processing apparatus 10 according to the present embodiment, in a case in which there is a possibility that the abnormality occurs in the physical condition of the user, the web search for useful information for the health management of the user is performed, so that it is possible to reduce the burden on the web search by the user.

In addition, the information processing apparatus 10 according to the present embodiment performs the first web search using the word input as the input information as the first search keyword, and further performs the second web search using the relation word related to the word input as the input information as the second search keyword. The information processing apparatus 10 performs the process of displaying the result of the web search for each of the first web search and the second web search. As described above, with the information processing apparatus 10 according to the present embodiment, the web search is also performed using the relation word in addition to the word directly input as the input information, so that it is possible to present a lot of information requested by the user without imposing the burden on the user, such as repeatedly performing the web search while changing the search keyword.

In addition, the information processing apparatus 10 according to the present embodiment performs the third web search using the text generated based on the biological information as the third search keyword. As described above, with the information processing apparatus 10 according to the present embodiment, in a case in which the measurement value shows an abnormal value as the biological information, the search keyword corresponding to the measurement value is generated and the web search is performed, so that it is possible to reduce the burden on the web search by the user.

In addition, the information processing apparatus 10 according to the present embodiment performs the process of classifying the results of the web search into the plurality of categories and displaying the classified results. As described above, with the information processing apparatus 10 according to the present embodiment, the results of the web search are classified into the plurality of categories and displayed, so that it is possible to easily and quickly access the information requested by the user.

In the embodiment described above, as a hardware structure of processing units that execute various processes, such as the input information acquisition unit 11, the biological information acquisition unit 12, the analysis unit 13, the search processing unit 14, the display processing unit 15, the first search processing unit 31, the relation word derivation unit 32, the priority determination unit 33, the second search processing unit 34, the search keyword generation unit 35, and the third search processing unit 36, the following various processors can be used. The various processors include, for example, a programmable logic device (PLD), such as an FPGA, which is a processor of which a circuit configuration can be changed after manufacture, and a dedicated electrical circuit, such as an application specific integrated circuit (ASIC), which is a processor having a dedicated circuit configuration designed to execute a specific process, in addition to the CPU and the GPU which are general-purpose processors that execute software (program) to function as various processing units as described above.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. A second example thereof is a form in which a processor that realizes all of the functions of a system including the plurality of processing units with one integrated circuit (IC) chip is used, as represented by a system on chip (SoC). In this way, various processing units are composed of one or more of the various processors as the hardware structure.

Further, more specifically, an electric circuit (circuitry) obtained by combining circuit elements, such as semiconductor elements, can be used as the hardware structure of the various processors.

In addition, in the embodiment described above, the aspect has been described in which the search program 110 is stored (installed) in advance in the non-volatile memory 103, but the disclosed technology is not limited to this. The search program 110 may be recorded on a recording medium, such as a compact disc read only memory (CD-ROM), a digital versatile disc read only memory (DVD-ROM), or a universal serial bus (USB) memory, and then provided. In addition, the search program 110 may be downloaded from an external device via a network.

What is claimed is:

1. An information processing apparatus comprising:
at least one processor,
wherein the processor performs a process of
acquiring input information, which is information input to the information processing apparatus, and biological information of a user,
performing a web search based on at least one of the input information or the biological information in a case in which it is determined that an abnormality occurs in a physical condition of the user based on at least one of the input information or the biological information, and
displaying a result of the web search.

2. The information processing apparatus according to claim 1,
wherein the input information includes a search keyword input to a search engine.

3. The information processing apparatus according to claim 1,
wherein the input information includes a word transmitted or sent to a specified or unspecified party.

4. The information processing apparatus according to claim 1,
wherein the input information includes a word uttered by the user in a conversation with a chatbot operated on the information processing apparatus.

5. The information processing apparatus according to claim 1,
wherein the processor performs a process of
performing a first web search using a word input as the input information as a first search keyword, and a second web search using a relation word related to the word input as the input information as a second search keyword, and
displaying the result of the web search for each of the first web search and the second web search.

6. The information processing apparatus according to claim 5,
wherein the processor derives the second search keyword by using an ontology database in which the first search keyword and the second search keyword are associated with each other.

7. The information processing apparatus according to claim 1,
wherein the processor performs a process of
performing a third web search using a text generated based on the biological information as a third search keyword, and
displaying a result of the third web search.

8. The information processing apparatus according to claim 1,
wherein the processor performs a process of classifying the results of the web search into a plurality of categories to display the classified results.

9. An information processing method executed by at least one processor provided in an information processing apparatus, the method comprising:
acquiring input information, which is information input to the information processing apparatus, and biological information of a user;
performing a web search based on at least one of the input information or the biological information in a case in which it is determined that an abnormality occurs in a physical condition of the user based on at least one of the input information or the biological information; and
displaying a result of the web search.

10. A non-transitory computer-readable storage medium storing an information processing program causing at least one processor provided in an information processing apparatus to execute a process comprising:
acquiring input information, which is information input to the information processing apparatus, and biological information of a user;
performing a web search based on at least one of the input information or the biological information in a case in which it is determined that an abnormality occurs in a physical condition of the user based on at least one of the input information or the biological information; and
displaying a result of the web search.
